(19) United States
(12) Patent Application Publication
(10) Pub. No.: US 2006/0034877 A1
(43) Pub. Date: Feb. 16, 2006

(54) Cationic lotion

(76) Inventor: Artus L. Satterfield, Irvington, NJ (US)

Correspondence Address: Artus L. Satterfield, 221 19th Ave., Irvington, NJ, 07111, US

(21) Appl. No.: 10/919,455

(22) Filed: Aug. 16, 2004

(51) Int. Cl. A61K 8/02

(52) U.S. Cl. 424/401

(57) ABSTRACT

Lotion made with saturated sodium chloride water and monohydroxy plus polyhydroxy alcohols to relieve stiffness in skin around knee joints.

This invention relates to skin tissue elasticity

### BACKGROUND

### Description of Prior Art

Skin lotions are used for cosmetic affects

### OBJECT AND ADVANTAGE

Cationic lotion penetrates hard dry skin relieves pressure around joints, especially the knees.

### DESCRIPTION

Sodium chloride is dissolved in water to the point of saturation. The saturated water is decanted and mixed with monohydroxy and polyhydroxy alcohols to produce a lotion.

### APPLICATION

Cationic lotion is applied to dry and hard skin with the fingers palm of hands

### SUMMARY, RAMIFICATIONS AND SCOPE

Tightness in the knees becomes painful and restricts the range of motion. Cationic lotion relieves the pain and increases the range of motion.

1. Skin lotion for hard and dry skin that relieves pressure around knee joints
